(19) United States
(12) Patent Application Publication
(10) Pub. No.: US 2026/0209264 A1
(43) Pub. Date: Jul. 23, 2026

(54) METHOD AND SYSTEM FOR DRYING OF PROTEINS AS WELL AS FUNCTIONALIZED PROTEIN OBTAINED

(71) Applicant: GEA Process Engineering A/S, Søborg (DK)

(72) Inventor: Sarah Veelaert, Søborg (DK)

(73) Assignee: GEA Process Engineering A/S, Søborg (DK)

(21) Appl. No.: 19/140,861

(22) PCT Filed: Dec. 20, 2023
(86) PCT No.: PCT/EP2023/086910

§ 371 Date: Jun. 18, 2025

(30) Foreign Application Priority Data
Dec. 21, 2022 (EP) 22215565.7

(51) Int. Cl. C07K 1/14; A23B 2/00; A23B 2/93; A23J 3/14; A23J 3/16; C07K 1/107; F26B 3/12

(52) U.S. Cl. CPC C07K 1/145; A23B 2/001; A23B 2/93; A23J 3/14; A23J 3/16; C07K 1/1077; F26B 3/12

(57) ABSTRACT

A method and system are provided for drying of non-mammalian proteins. Protein is extracted and subjected in one single operation to a steam infusion treatment under high shear conditions and spray drying. In a further aspect, functionalized, dried, non-mammalian protein powder obtained by said method and showing high viscosity when redispersed in water, is provided.

### TECHNICAL FIELD

The present invention relates to drying of non-mammalian proteins.

### BACKGROUND ART

Proteins are the building blocks of living organisms and play important roles in almost all biological processes. The use of concentrated proteins is of interest for food, feed and medical applications alike, and in recent years the employment also of e.g. plant-derived proteins and fermentation derived proteins has gained momentum due to their versatile applications. The physiochemical and structural properties as well as the functional attributes of plant-based proteins are of great interest to the food and processing industries, and a range of proteins are utilized or investigated not only as foodstuffs in their own right but also as a functional ingredient e.g. for texturizing agents, e.g. for meat and dairy substitute products, and as viscosifiers for improved mouthfeel in beverages or for improved oil/fat binding capacity.

When used in the latter applications, wherein the proteins play an important role in defining the texture of the final product, they are often applied as concentrates or isolates, which have been functionalized for increased viscosity, gelation and fat- or water-binding capacity.

“Functionalization” is generally referred to in the art as a complex process involving denaturation, dissociation-association and aggregation of the protein, and may in different contexts be accomplished by physical means such as heating or pressurization, by chemical means, or by a combination of physical and chemical means. With a view to increased solubility and digestibility, proteins may be hydrolyzed by enzymatic or chemical methods, which break down large and/or complex protein molecules to smaller entities. “Protein isolates” generally refer to proteins, which have undergone intense processing, e.g. by pH treatments and mechanical separation, to remove a very large proportion of non-proteinaceous constituents such as carbohydrates and fats.

In the context of the present application, “protein concentrates” are taken to encompass also “protein isolates”.

Apart from the question of viscosity, gelation and fat- or water-binding capacity, another demand on proteins used for modifying the rheology of foodstuffs relate to their microbiological properties. For food safety reasons, a reduction of the inherent microbial load of the respective proteins may be required to remove any risk related to the action of pathogenic bacteria. Therefore, edible proteins are often concentrated by means of a drying procedure involving a preceding step of heating to reduce bacterial or spore counts, such as to remove pathogenic bacteria.

Prior to concentration, proteins must be extracted and separated from a raw material. When extracting microbially-produced or plant-based protein, alkaline extraction is typically brought about by adding a flour or meal of protein-containing material to a lye. A concentrated protein fraction is then separated from a liquid fraction either by acid precipitation and decanting or by membrane filtration. The separated concentrated protein fraction is dispersed in a solvent, normally water, and pH adjusted to above the isoelectric point of the protein in order to have it in a nearly dissolved state. Generally, the solubility of nonhydrolyzed protein is around 50 to 80% at a pH within the range of 6 to 8.5, and it will present itself as a colloidal dispersion known as a protein liquid feed.

Conventionally, the diluted liquid feed is then heat treated, primarily to reduce the microbial load but also to functionalize the protein. The heat treatment process may be direct, such as by steam injection or steam infusion, or indirect, such as by means of a heat exchanger, and will result in a viscosity increase or gel formation depending on the type, purity, concentration, and pH of the protein dispersion. To stop the heat treatment, the liquid feed must be cooled down, such as by flashing off steam in an expansion vessel. At this point, some proteins are prone to start gelling due to reduction of temperature and loss of pressure, complicating further handling as described in the following.

The heat-treated liquid feed conventionally is pumped, possibly via an intermediate buffer tank, to a pre-pressure pump and a high-pressure pump of a spray dryer, and in the spray dryer the liquid feed is atomized by means of a high-pressure nozzle.

Here, a dilemma appears; on one hand, the functionality of the protein after drying is enhanced, so that the more the viscosity of the protein liquid feed is increased during the heat treatment the better, but on the other hand the liquid feed gets more and more difficult to pump and atomize in the spray dryer as it thickens. Hence, to forestall excessive gelling, the protein concentration must be kept below a certain threshold by dilution of the liquid feed. In turn dilution requires a subsequent evaporation and treatment of water in the drying phase, which is energy consuming.

In European patent application EP 0032296, a process for cooking or gelatinizing starch is disclosed, wherein steps of heating and atomizing the starch (possibly mixed with other ingredients) are brought together within the confines of a so-called cooking cap. The applied process is explicitly adapted for preserving integral granules of starch by minimal shearing and heat treatment, and the cold starch slurry used as a starting material has a very low viscosity, similar to water (1 cP). Further, microbial load is much less of an issue in starch than in proteins.

In US2014/0023772A1 a process aiming at increasing the production capacity of a powdered composition from a liquid composition comprising fat, protein or both is described. In a first atomizing step, a liquid feed is fed through an inlet nozzle, preferably a pressure nozzle, into a mixing chamber comprising steam and gas. Then follows a second atomizing step, wherein the atomized mixture of liquid composition, steam and gas leaves the mixing chamber through an outlet nozzle, preferably into a drying chamber. There is no disclosure of the pressure of the feed but the mixing chamber pressure is disclosed to be 2 to 10 bar. The disclosed benefit of the method is increased production capacity while not compromising the properties of the final powder. In terms of properties focus is on uniform bulk density of infant formula powder. However, the disclosure does not address functionalization and decreased microbial load of proteins nor does it address a more energy efficient method.

The prior art solutions fall short of addressing the specific challenges connected to the viscous nature of protein feeds to be dried as set out in the above. Even before any heating step has been applied, the viscosity of colloidal protein dispersions will often be in the range of 100 to 1,000 cP (mPa*s), increasing after heating typically up to 1,000-5,000 cP (mPa*s), but even higher, up to 100,000 cP (mPa*s), when the dispersions are allowed to gel at room temperature (measured at shear ratios of 100 s⁻¹).

With the increasing interest in non-mammalian protein sources there is an increased demand for finding methods that are able to provide texturized protein in a simple, cost and energy efficient manner while observing the requirements for reduced microbial load for products in particular to be used for consumption.

### SUMMARY OF THE INVENTION

In view of the above, it is an object of the present invention to provide a technical solution for solving one or more of the above problems by combining concentration and functionalization of proteins in a single operation of heating, shearing and drying, while at the same time reducing microbial load, energy consumption and water consumption.

To meet this object, a method is provided for drying non-mammalian protein, the method comprising the steps of:
a) providing a, preferably comminuted, material containing the non-mammalian protein, such as native non-mammalian protein;
b) extracting and separating protein from said material to obtain a protein enriched fraction of extracted protein, or otherwise obtaining a liquid, colloidal or semi-solid protein enriched fraction from said material;
c) optionally dispersing, and pH adjusting the protein enriched fraction of extracted protein or the otherwise obtained protein enriched fraction to obtain a protein liquid feed, preferably the pH adjustment is above the isoelectric point of the protein;
d) providing a steam flow to a steam infusion fluid nozzle mounted in a spray dryer, thereby generating a steam atmosphere in an infusion chamber of the steam infusion nozzle;
e) providing the protein liquid feed to the steam infusion nozzle;
f) introducing the protein liquid feed into the steam atmosphere of the infusion chamber of the steam infusion nozzle, at a pressure of at least 5 barg, thereby primary atomizing and heating the protein liquid feed in a process of primary atomization;
g) allowing the atomized, heated protein to directly enter a drying chamber of the spray dryer through an opening in the infusion chamber in a process of secondary atomization, the drying chamber being provided with a drying gas; and
h) drying the atomized, heated protein in the spray dryer to obtain a dried protein.

By combining heat treatment and spray drying of the protein as consecutive steps in a single procedure, considerable savings of water, energy and space are obtained, as no separate installation for heat treatment is needed. Hence, unlike prior art methods a more simple and cost effective method is provided that provides at least comparable, but also improved texture properties of the final product depending on the intended purpose.

In addition, contrary to prior art methods, in the present method, the employment of higher feed concentrations is possible since viscosity only develops inside the infusion chamber of the steam infusion nozzle, after which the protein liquid feed is immediately atomized and dried without further need of handling a high viscous liquid protein feed. Further, no cooling step is required after the heat treatment, since the protein liquid feed immediately cools down when atomized in the dryer, where it is being dried, and excess steam is flashed off during the atomization.

In the present context, “protein” should be understood to possibly encompass several classes of protein. For instance, protein in a pea seed mainly consists of albumins and globulins. Albumins are considered metabolic and enzymatic proteins, while the globulins act as storage proteins for the seed. “Non-mammalian” proteins in the current application should be taken to also include analogues to mammalian proteins produced by genetically engineered microorganisms.

The high pressure in the nozzle upstream of its infusion chamber provides for the necessary high shear that is necessary in order to reduce the viscosity of the protein within the nozzle. The protein liquid feed is introduced into the steam atmosphere at the infusion chamber of the steam infusion nozzle at a pressure of at least 5, 10, 50, 100, 150, 200, 250, or 300 barg.

It is preferred that the protein liquid feed is introduced into the steam atmosphere at the infusion chamber of the steam infusion nozzle at a pressure of at least 50 barg, more preferred at least 100 barg.

Generally, it is believed that the reduction of microbial load and the degree of functionalization will depend on the interaction between the products of the primary atomization and the steam environment. Without the wish to be bound by theory it is currently believed that, all other factors being equal, smaller droplets or thinner films will yield a better interaction due to a better heat transfer, and to this end, and depending on the task at hand, a certain minimum value of the pressure at which the protein liquid feed is introduced into the steam atmosphere of the infusion chamber of the infusion nozzle will often be required.

It is understood that a “steam infusion nozzle” as used herein denotes an atomization nozzle such as a two-fluid nozzle, wherein an atomization gas is used to atomize a liquid feed. When in the present context mention is made of “primary atomization” within the steam infusion chamber of the steam infusion nozzle, this should be taken to encompass not just atomization sensu stricto but also related processes such as pre-filming, shearing, formation of a film and/or drops with a large surface area, and violent mixing. Nozzles configured for further fluids are also included, such as a three-fluid nozzle, which in addition to steam flow and protein liquid feed is supplied with compressed atomization gas.

In one embodiment, the protein liquid feed obtained in step c) is pre-heated to a temperature of 30 to 140° C., preferably 35 to 90° C., more preferred 40 to 80° C., even more preferred at least 40° C., such as 40 to 80° C., and more preferred 45° C. to 65° C., and even more preferred 50 to 60° C. prior to being provided to the nozzle. Depending on the protein to be dried, pre-heating at such temperatures does not initiate unwanted gelling and/or functionalization, and furthermore including a preheating step reduces the heat load and the amount of steam to be used in the nozzle, thereby further reducing the operational costs of the method of the invention.

According to a specific embodiment, the protein liquid feed is provided to the nozzle in step e) at a protein solid concentration of 8 to 30% (w/w), preferably 15 to 25% (w/w), most preferred 17 to 22% (w/w). In such embodiments the protein has typically not been hydrolyzed.

In an embodiment the pH of the feed is in the range of 5 to 8. pH in the lower end provides for a relatively stable product.

In another embodiment the pH of the feed is in the range of 7 to 9, preferably 8.5. It was surprisingly found that when pH increases the textures slowly starts to change and it was found that when pH is above 8 the effect was pronounced. Hence, if there is a need for a functionalized protein where the texture changes upon heating it is preferred that the pH of the feed is in the higher range. In particular embodiments, wherein the protein has been subjected to prior full or partial hydrolyzation, the protein liquid feed is provided to the nozzle in step e) at a protein solid concentration exceeding 30, 40, or 50% (w/w), such as 35 to 50% (w/w).

The protein liquid feed may optionally be provided at a feed rate of 30 to 1500 kg/h, preferably 100 to 900 or more preferably 250 to 750 kg/h.

Preferably, the steam flow is provided to the nozzle at a pressure of 1 to 16 barg, preferably 4 to 13, most preferred 5 to 10 barg.

The steam flow may optionally be provided at a feed rate of 15 to 1200 kg/h, such as 60 to 750 or preferably 80 to 600 kg/h.

In a preferred embodiment, the steam flow and the protein liquid feed are provided to the nozzle in a weight proportion of steam:liquid feed of 1:10 to 10:1, preferably 1:7 to 3:1, more preferred 1:6 to 2:1 and even more preferred 1:2 to 1:1 and most preferred 1:3 to 1:2.

The ratio of steam is important to achieve the desired effect, since if too little steam is provided, the protein feed will drip down through the infusion chamber opening without atomizing. On the other hand, if too much steam is supplied, the process will be less efficient and more energy will be needed for drying the protein.

In one embodiment, the temperature in the infusion chamber during the process of primary atomization is in the range of 120 to 200° C., preferably 150 to 190° C., more preferred 155 to 185° C., most preferred 160 to 180° C.

According to a specific embodiment, the spray dryer is of a type, wherein the protein is further dried in an integrated or external fluid bed, or in an integrated fluid bed as well as in an external fluid bed.

In an embodiment the dried protein may be further processed. A further processing of the dried protein may comprise agglomeration and/or lecithination of the protein.

It is contemplated that the means of extracting or separating the protein enriched fraction from the material in step b) comprises one or more of alkaline extraction, acid precipitation, membrane separation, ultrafiltration or a combination of micro- and ultrafiltration or acid precipitation. Among said means, acid precipitation, membrane separation, ultrafiltration or a combination of micro- and ultrafiltration could also be regarded as purification procedures. When using acid precipitation, the protein enriched fraction is obtained by washing out undesirable small molecules from said material at a pH near the isoelectric point and keeping the precipitate or remaining protein enriched fraction. The liquid feed should be free of particles that would block the nozzle, such as fibers. Also, it is contemplated that the liquid feed may be diluted if necessary.

In a specific embodiment, the extracted protein is a pea protein; the protein liquid feed in step c) is adjusted to pH 6.5 to 8; the protein liquid feed obtained in step c) is pre-heated to 40 to 80, preferably 45 to 65° C.; the protein liquid feed in step e) is provided to the steam infusion nozzle at a protein solid concentration of 18 to 22% (w/w); the steam flow and the protein liquid feed are provided to the steam infusion nozzle in a weight proportion of steam to liquid feed of 1:7 to 3:11, preferred 1:6 to 2:1; the protein liquid feed in step f) is introduced into the steam atmosphere of the infusion chamber of the steam infusion nozzle at a pressure of 150 to 270 barg, preferably 250 to 270 barg; and the temperature in the infusion chamber during the process of primary atomization is in the range of 160 to 185° C. The opening of the infusion chamber preferably has a diameter of 2 to 8 mm, more preferred 3 to 7 mm depending on the protein liquid feed rate, and the distance from the end of a liquid feed channel of the steam infusion nozzle to the opening of the infusion chamber preferably is 10 to 60 mm, such as 25 to 40 mm.

In an alternative embodiment, the extracted protein is a soy protein; the protein liquid feed in step c) is adjusted to pH 6.5 to 8; the protein liquid feed obtained in step c) is pre-heated to 45 to 65° C.; the protein liquid feed in step e) is provided to the steam infusion nozzle at a protein solid concentration of 8 to 14% (w/w); the steam flow and the protein liquid feed are provided to the steam infusion nozzle in a weight proportion of steam to liquid feed of 1:1.0 to 1:5; the protein liquid feed in step f) is introduced into the steam atmosphere of the infusion chamber of the steam infusion nozzle at a pressure of 180 to 300 barg, preferably 230 to 300 barg; and the temperature in the infusion chamber during the process of primary atomization is in the range of 155 to 185° C.

The opening of the infusion chamber preferably has a diameter of 2 to 8 mm, more preferred 3 to 7 mm, such as 5 or 6 mm depending on the protein liquid feed rate, and the distance from the end of a liquid feed channel of the steam infusion nozzle to the opening of the infusion chamber preferably is 20 to 50 mm, more preferred 35 to 45 mm.

In a second aspect of the invention is provided a system for drying a non-mammalian protein, the system comprising a spray dryer having a drying chamber, at least one drying gas inlet, and at least one product outlet; at least one steam infusion nozzle configured for generating a protein liquid feed spray into the spray dryer; wherein the steam infusion nozzle comprises a first channel and a second channel, the first and second channel being in fluid communication with an infusion chamber attached to the steam infusion nozzle, the first channel being connected to a supply of a liquid feed of the non-mammalian protein, the second channel being connected to a supply of steam, and the infusion chamber being provided with an opening facing the drying chamber of the spray dryer.

The opening of the infusion chamber preferably has a diameter of 2 to 10 mm, such as 3 to 8 mm, more preferred 4 to 7 mm, and the distance from the end of the first channel to the opening of the infusion chamber preferably is 10 to 100 mm, such as 25 to 80 mm, more preferred 25 to 45 mm, such as 25 to 35 mm or 35 to 45 mm.

In general, for a given protein liquid feed to be treated, the diameter of the opening of the infusion chamber as well as the distance from the end of the first channel to the opening would be smaller for a lower protein liquid feed rate. The diameter of the opening affects the speed through the opening and the secondary atomization properties, while, if the diameter and all other parameters are kept unchanged, an increased distance between the first channel and the opening will yield an increased volume of the infusion chamber and thus an increased residence time of the product of primary atomization therein.

In a third aspect of the invention, a dried, non-mammalian protein powder is provided, said protein powder being functionalized and/or showing bacteria or spore counts reduced to a stipulated level, obtained by the method according to the invention.

In one embodiment, the dried, non-mammalian protein powder is functionalized pea protein powder, and a dispersion of 14% (w/w) of said powder in water shows, after being heated in the course of 3 minutes from 50 to 100° C., kept at 100° C. for 2 minutes and then cooled to 50° C., a level of viscosity at 50° C. of more than 2500 cP (mPa*s).

Preferably, if the viscosity of the pea protein powder dispersion decreases during said temperature treatment, it does so by no more than 20%, such as no more than 15% or no more than 10% (cP at 50° C.).

Preferably, the dispersion of the pea protein powder after said temperature treatment shows a viscosity, which is at least 30, such as 30 to 350% (cP at 50° C.) higher than the viscosity of a dispersion of 14% (w/w) of a powder of the same pea protein, but wherein the protein has been subjected instead to conventional pasteurization at a temperature ranging from 75 to 95° C., for 10 minutes to 1 hour, e.g. in a heat exchanger, and wherein both dispersions are subjected to the same temperature treatment and test conditions during the measurement of viscosity.

The functionalized, dried, pea protein powder characterized by one or all of the features above is obtainable by the method of the invention.

In an alternative embodiment, the dried, non-mammalian protein powder is functionalized soy protein, and a dispersion of 12.5% (w/w) of said powder in water, 5 minutes after preparation of said dispersion, shows a level of viscosity at 25° C. of more than 13000 cP.

Preferably, the dispersion of the soy protein powder shows a viscosity, which is more than 30% (cP at 25° C., 5 minutes after preparation) higher than the viscosity of a dispersion of 12.5% (w/w) of a powder of the same soy protein, but wherein the protein has been subjected instead to conventional steam injection at 135° C. and subsequent, detached step of spray drying as commonly known in prior art production processes of soy protein isolates or concentrates, and wherein both dispersions are subjected to the same test conditions.

In one method of measuring the viscosity, the protein dispersion is prepared at room temperature, with 1 min high shear mixing, after which it is allowed to rest at room temperature for 6 h; and then the viscosity is measured using standard methods as detailed below.

The functionalized, dried, soy protein powder characterized by one or all of the features above is obtainable by the method of the invention.

The viscosity is measured using standard methods, such as in a Rapid Visco Analyzer (RVA apparatus) obtainable i.a. from Perkin Elmer.

Other presently preferred embodiments and further advantages will be apparent from the subsequent detailed description and drawings.

### BRIEF DESCRIPTION OF DRAWINGS

In the following description, embodiments of the invention will be described with reference to the schematic drawings, in which:

FIG. 1 is a cross-sectional view of a steam infusion nozzle with its attached infusion chamber usable according to an embodiment of the invention;

FIG. 2 shows a system according to an embodiment of the invention; the location of the central part of the steam infusion nozzle illustrated in FIG. 1 is indicated by a dotted circle;

FIGS. 3A and 3B are perspective views showing two embodiments of the infusion chamber released from the steam infusion nozzle usable according to the invention;

FIG. 4 is a schematic illustration of the process according to the invention.

FIGS. 5 and 6 are graphs showing the viscosity as a function of time of functionalized proteins prepared according to embodiments of the invention as compared to prior art methods.

### DETAILED DESCRIPTION

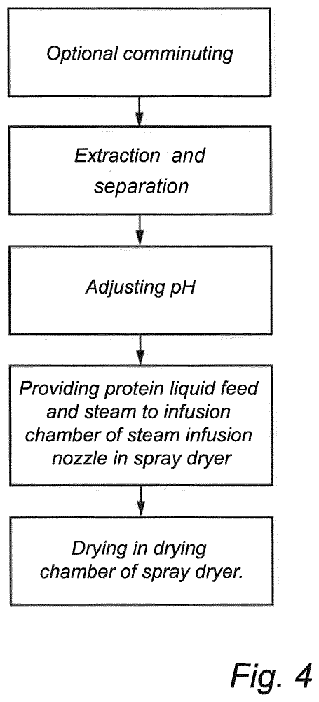

For a brief overview of the method according to the invention, reference is made to FIG. 4, wherein method steps are summarized as: optional comminuting; extraction and separation; adjusting pH; providing protein liquid feed and steam to an infusion chamber of a steam infusion nozzle in spray dryer; and drying in drying chamber of spray dryer.

Before turning to a detailed account of the respective steps, a description of the starting materials, the general principles and the products of the method of the invention will be given in the following.

According to the invention, non-mammalian protein encompasses protein originating from plants, seaweed, fungi, micro-organisms, genetically engineered microorganisms producing analogues to naturally occurring proteins, including mammalian proteins. Further, while presented as directed to non-mammalian protein, the method of the invention is also applicable to mammalian proteins. Proteins derived from fish and marine or terrestrial invertebrates such as insects, crustaceans, molluscs, shellfish and jellyfish are also encompassed, as are proteins derived from animal skin, bones and hair. In preferred embodiments non-mammalian protein is plant protein such as protein from legumes, for instance peas, chickpeas, cowpeas, peanuts, lupin, lentils, soy beans, mung beans, fava beans, other beans; cereals, such as wheat, rice, sorghum, minor millets, maize, barley, rye and oat; pseudo-cereals such as amaranth, buckwheat and quinoa; other seeds such as rape seeds, canola, sunflower, pumpkin, sesame, flaxseed, cottonseed; proteins derived from vegetative plant parts, such as leaves, sprouts, stems or roots of e.g. grasses, lucerne, clover, saltwater or freshwater algae; tubers such as potato or yams.

As an example of a suitable microorganism high in protein by nature, spirulina (Arthrospira spp.) could be mentioned.

Especially preferred non-mammalian protein are pea, soy, chickpea, fava bean, mung bean, rice, barley, oat, sunflower, canola, potato, and grass.

According to the invention, functionalized or texturized protein means protein that has been subjected to one or more of denaturation, dissociation-association and aggregation of the protein. Already during alkaline treatment and subsequent acid addition of the extraction and separation, some denaturation of protein will occur.

Protein dispersions as those applied as liquid, colloidal or semi-solid feed in the present invention often show the characteristics of a non-Newtonian fluid, meaning that they show a clear change in response to shear force. A shear thinning effect, be it time-dependent (thixotropic) or instant, may thus be imparted by the forcing of the liquid feed through the steam infusion nozzle at high pressure. On the other hand, the heat treatment, to which the protein liquid feed is also subjected in the steam infusion nozzle, generally tends to increase its viscosity. In the method of the invention, these oppositely acting processes are coordinated and taken advantage of.

According to the invention the solvent for the dispersion generally comprises, consists or essentially consists of water. Partly or fully non-aqueous solvents may play a role if compatible with safety regulations in relation to the intended use of the processed protein.

According to the invention, the heating medium fed to the steam nozzle is preferably saturated steam but can also be unsaturated steam or supersaturated steam. Partly or fully non-aqueous vapors may also come into question as heating media if compatible with safety regulations in relation to the intended use of the processed protein.

The resulting texturized protein has many edible applications, be it directly or as a starting material for further processing, and may, depending on its specific properties, be used e.g. as a food supplement, a food texturizer or stabilizer, an edible coating, or a hydrogel, and may also have non-edible applications, for instance as an adhesive.

When used as food supplements, the amino acid composition of the proteins is the most important factor determining their quality. A major difference between animal and plant proteins is that the latter generally are much lower in lysine. However, proteins from legumes such as peas and especially soy are relatively higher in lysine but lower in sulfuric amino acids than other plant proteins, and some nutrition experts have suggested a combination of legume and grain proteins in the diet as a fortification strategy to fulfil the required demand for essential amino acids.

As a texturizer the protein is, as customary in the art, used to give viscosity, form gels, bind water or fat, give elastic properties or make a foam.

In their capacity as amphiphilic polymers, plant proteins may be suitable for stabilizing conventional food-based emulsions. As the protein particles adsorb onto the interface between the water phase and the oil phase of an emulsion, a stabilized, so-called Pickering emulsion is formed. In general, versatile applications of plant proteins as viscosifiers or texturizers for food and beverages for human or animal consumption are contemplated or already realized.

Edible coatings may be in the form of thin layers applied to food items to block the entry of moisture, oxygen and movements of solutes without altering the taste and texture of the food item. Especially globular plant proteins, such as wheat gluten, are highly appropriate for use in the production of edible coatings.

Plant protein and peptide-based hydrogels are crosslinked polymeric meshworks prepared from globular proteins and capable of for example controlled released of encapsulated substances such as drugs and bioactive compounds into the surrounding environment.

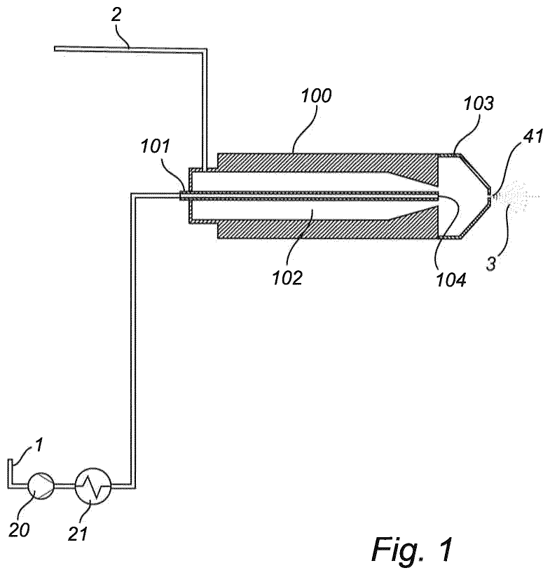

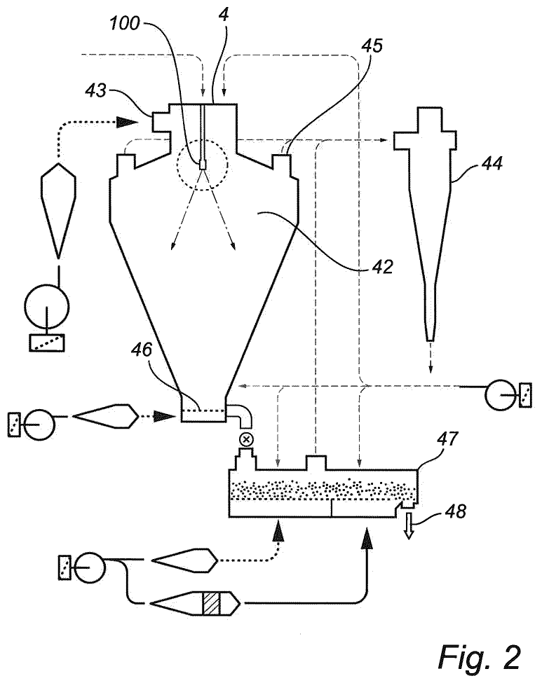

The method of the invention will now be described in further details with reference to FIGS. 1 and 2.

FIG. 1 shows a process scheme of the functioning of the steam infusion nozzle according to an embodiment of the invention. A protein liquid feed 1 is provided to the steam infusion nozzle 100 by way of at least one pump 20. A heat exchanger 21 is provided to optionally pre-heat the liquid feed 1. Subsequently, the liquid feed 1 is supplied by means of a high-pressure pump (not shown) to a first channel 101 of the steam infusion nozzle, and a steam flow 2 is supplied to a second channel 102 of the steam infusion nozzle.

In the first steps of providing the protein liquid feed, standard methods may be used.

The material, from which the protein should be extracted, will often need to be cleaned from impurities, such as by washing or air blowing. In the case of plant seeds, further steps of dehulling, possibly crushing to produce an oil side stream, milling and sifting will generally be required to produce a flour or meal or flakes for further processing.

When using a so-called protein isolate approach, extraction of the protein fraction from the flour or flakes is done at alkaline conditions at a pH of 8 or more. Subsequently, precipitation takes place at acid conditions around pH 4.5, but, alternatively, precipitation could be replaced by membrane filtration, for which there is no need to work at acidic pH. For the next step is then either received a pH 4.5 curd as an underflow from a mechanical separation device such as a decanter or centrifuge, or a retentate from a membrane filtration process.

Depending on the desired functionality of the final protein product, dilution and pH adjustment is then performed to obtain a protein liquid feed at pH 5 to 9, which is typically above the isoelectric point of the protein. In some embodiment the pH is in the range of 5 to 8 and in other embodiments the pH is on the range of 7 to 9, preferably 8.5.

Alternatively, the protein enriched fraction could be separated and purified from the flour or flakes by washing out unwanted smaller sugars from the flour or flakes at a pH near the isoelectric point; this may be denoted the protein concentration approach.

The method of providing the protein liquid feed does not substantially affect the subsequent drying step.

The thus obtained protein liquid feed 1 is provided to the first channel 101 of the nozzle 100 by means of the pump 20. The infusion chamber 103 of the nozzle is filled with steam 2 by a continuous supply of steam from the second channel 102 at a chosen temperature and steam pressure, and the protein liquid feed is atomized from the end 104 of the first channel 101 and finely dispersed into the steam environment. In some instances, proper atomization may not be attained at this stage, but then at least a process of pre-filming and shearing with a large surface area and violent mixing will be brought about. The steam does not have a “direction” as such but will try to escape the orifice 41 of the infusion chamber 103 due to the high pressure. Upon leaving the orifice 41 of the infusion chamber 103, the steam with entrained protein liquid feed brings about a secondary atomization 3.

In a variation of the method, the protein liquid feed is preheated, in a heater 21, before being fed to the nozzle 100. Depending on the protein to be dried, pre-heating at such temperatures does not initiate unwanted gelling and/or functionalization before being fed to the nozzle 100, and furthermore, having a pretreatment step reduces the heat load and the amount of steam to be used in the nozzle 100, thereby further reducing the operational costs of the method of the invention.

In some applications, the main purpose of the heating is to reduce the microbial load, whereas the conformation of the protein should often remain as native as possible. For achieving this, ultra-high temperatures in the infusion chamber for a very short time are often preferred in order to maximise the destruction of microorganisms while minimising any other denaturation of the proteins.

The method and system allow for a heat treatment at higher temperatures than normal UHT treatment (2 to 5 seconds at 135° C.) without altering the protein conformation. According to the invention, temperatures of up to 180° C. can be reached at a steam pressure of about 9.5 barg for a very short period of time.

The common Z values for the thermal inactivation of microorganism are around 5-6° C. up to 10° C. A Z value of 10° C. means that if the heating temperature would be increased by 10° C., a 10 times lower residence time would be needed to achieve the same log reduction of the microorganism. So, this means that by increasing the temperature from a normal UHT condition at 135° C. to 175° C., the heating time can be reduced by a factor 104. Thus, if choosing a nozzle design that allows for operation at a steam temperature of 175° C., the infusion chamber would require much lower residence time in the order of 2 to 5×10⁻⁴ seconds while achieving a thermal inactivation of the microorganisms comparable to a conventional UHT treatment. Thus, the higher the temperature, the shorter the heating time should be, in order to avoid negative side effects of the thermal treatment. In this regard, heat treatments for e.g. 2 to 5×10⁻¹ seconds at around 145° C., 2 to 5×10⁻² seconds at around 155° C., 2 to 5×10⁻³ seconds at around 165° C., or 2 to 5×10⁻⁴ seconds at around 175° C. are contemplated.

Rapid heating and cooling steps are therefore an important benefit of the present invention, in particular when the main purpose is reducing the microbial load without altering the texture of the protein.

Rapid heating can be achieved by atomizing very fine droplets into a continuous flow of steam (infusion). With fine droplets the surface area of the droplets is maximized, and the heat transfer between the continuous steam and the droplets results in a uniform heating of the droplets inside the infusion chamber.

The rapid cooling is achieved by flashing off the steam by lowering the pressure. The advantage of the present invention is that the flashing off of the steam occurs during the secondary atomization out of the infusion chamber, into the drying chamber. Therefore, the difficulties in the prior art of letting the steam escape from the viscous gel, which is formed upon cooling, is avoided.

The pressure in the drying chamber 42, FIG. 2, is slightly below ambient pressure. A typical underpressure is around 0.49 to 0.98 mbarg (5 to 10 mm water column).

The secondary atomization is driven by the steam velocity through the opening 41 of the infusion chamber 103, which might be as high as sonic velocity. The higher the steam flow, the finer the atomized droplets 3 will be, and the faster the cooling of the droplets. Generally, at least 20% of the total steam flow (excess steam) will be used for the purpose of secondary atomization.

Hence, the steam has the dual role of 1) heating protein colloidal dispersion, whereby some vapour will condense; and 2) driving protein forward to effect secondary atomization in the drying chamber 42 of the spray dryer of the system 4, for which excess steam is required. In this context it should be noted that if too little steam is provided, the protein feed will drip down through the infusion chamber without atomizing. On the other hand, if too much steam is supplied, the process will be less efficient and more energy will be needed for drying the protein. In some cases, as little as 5-10% excess added steam will be sufficient for atomization due to flashing of water in the super heated protein dispersion.

Should a gel be formed during cooling, it will be formed within the individual atomized small droplets that are immediately dried when entering the drying chamber 42 of the spray dryer of the system 4 and hence it will not affect the processability of the liquid, since the gel is not formed while protein liquid feed is under pressure and being fed to the infusion chamber through the nozzle, and due to the high temperature in the infusion chamber.

In some embodiments, the dried protein may be further dried in a fluid bed, such as a standard fluid bed drier 46 integrated with the system as shown in FIG. 2.

The method of the invention provides dried, texturized/functionalized protein.

With reference also to FIG. 2, an embodiment of a system and infusion chamber according to the invention will be described in more details. According to the invention, the combined heating and drying is performed in a system 4 comprising a spray dryer with a drying chamber 42 and being equipped with a steam infusion nozzle 100 including an infusion chamber 103, cf. FIG. 1. The infusion chamber 103 may be an integral part of the nozzle 100, or it may be, preferably releasably, attached to the nozzle 100 so that the infusion chamber 103 may be exchanged depending on the protein to treat and the desired effect. Also, for cleaning purposes and with a view to easy exchange of the remainder of the nozzle 100 or components thereof, a releasable infusion chamber 103 is favourable. Protein and steam enter the infusion chamber 103 through the first 101 and second 102 channels, respectively, of the steam infusion nozzle 100. Excess steam is applied in order to atomize the protein 3 in a secondary atomization through the orifice 41 of the infusion chamber facing the drying chamber 42 of the spray dryer of the system 4.

The dried protein may be further dried in a fluid bed 46 and possibly also or alternatively in an external vibrating fluid bed drier 47 connected to the spray dryer 4. In the embodiment shown, the dried product exits via the product outlet 48, and the system further possesses a drying air inlet 43 and a drying air outlet 45, the latter being connected to a cyclone 44, which could be replaced by a bag filter. All equipment is standard equipment generally available in the art.

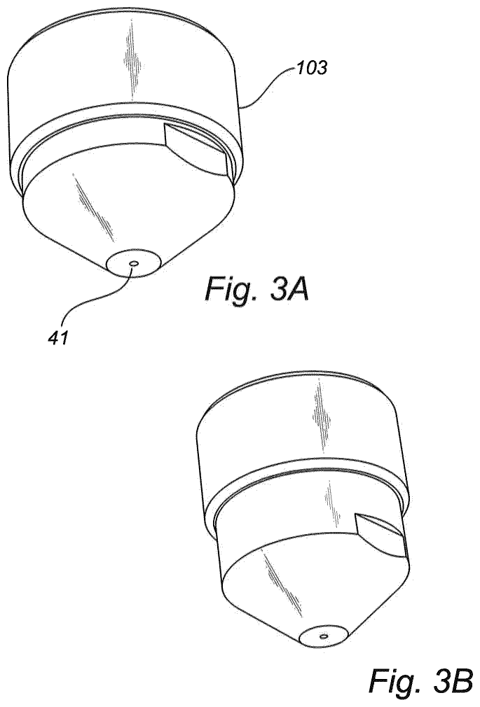

Referring again to FIG. 1, the infusion chamber 103 may vary in size and the diameter of the orifice 41 of the infusion chamber as well as the distance from the end 104 of the first channel 101 to the orifice 41 may vary as well. These parameters affect the time of residence of the protein in the infusion chamber 103 and thereby the reduction of microbial load and final functionalization of the protein. Thus, releasable infusion chambers of different lengths, compare FIG. 3A and FIG. 3B, may be employed for the specific task at hand.

### EXAMPLES

The effect of the method and operation of the system will now be illustrated with reference to the following non-limiting examples. The starting material is a protein liquid feed as provided in step c) of claim 1 by using standard methods as described above. the steps d and onwards are further detailed in the various examples as are properties of the feed (solid content and pH). As is known to the skilled person 1 cP is 1 mPa*s, hence any denotation of cP is the same in mPa*s.

### Example 1—Test with Pea Protein

The protein solid concentration was 19% (w/w) and the dispersion had a pH of 7. The dispersion was fed to the steam infusion nozzle at a rate of 136 kg/h and at a temperature of 45° C. The nozzle pressure upstream of the infusion chamber was 260 barg and testing was performed at three different conditions:

TABLE 1
test parameters

| Test no. | 1 | 2 | 3 |
|---|---|---|---|
| Steam pressure | 9 | 7 | 6 |
| (barg) | | | |
| Steam flow (kg/h), | 115 (1:1.18) | 92 (1:1.48) | 83 (1:1.64) |
| (steam to feed ratio) | | | |
| Temperature (° C.) | 180 | 170 | 165 |

The orifice of the infusion chamber had an opening diameter of 5 mm.

The resulting size of the final powder, measured as D₅₀ with a Malvern particle sizer, was 120 micron.

For evaluating the viscosity of the now functionalized protein, spray dried powders were redispersed in water at 14% w/w. The dispersions were mixed by hand and then heated from 50 to 100° C. in a RVA apparatus (Rapid Visco Analyzer) in the course of 3 minutes, kept at 100° C. for 2 minutes and then cooled to 50° C.

For comparison, a 14% w/w dispersion was also prepared from a powder based on the same protein liquid feed but having been processed in a conventional manner, i.e. by conventional pasteurization in a heat exchanger at approximately 85° C. for approximately 15 minutes with subsequent spray drying as a separate step.

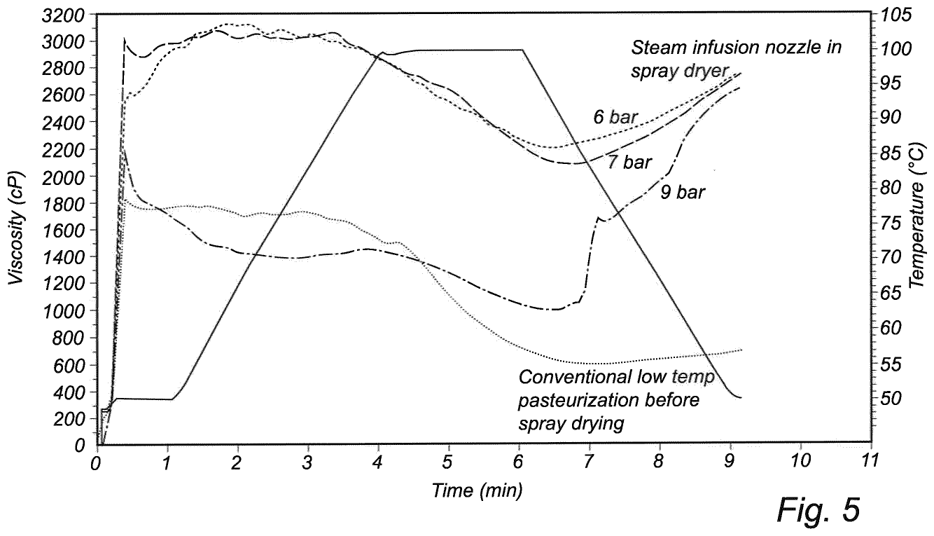

The viscosity at 50 rpm was measured before, during and after heating and cooling using the standard procedure of the Rapid Visco Analyzer (PerkinElmer). The results appear from FIG. 5, wherein also the heating and cooling profile is shown as an unbroken line. Viscosities at 50° C. before and after heating of the dispersion were as follows:

TABLE 2
viscosity results

| | Viscosity | Viscosity |
|---|---|---|
| | at 50° C. (cP) | at 50° C. (cP) |
| | before heating | after heating |
| Steam infusion nozzle | 1630 | 2646 |
| 9 barg (Test 1) | | |
| Steam infusion nozzle | 2996 | 2734 |
| 7 barg (Test 2) | | |
| Steam infusion nozzle | 2958 | 2745 |
| 6 barg (Test 3) | | |
| Conventional | 1779 | 673 |
| pasteurization | | |

Thus, viscosities at 50° C., after heating, of more than 2600 cP were found for dispersions of protein powder prepared according to the invention. This is almost 4 times as high a viscosity as that found for the dispersion of protein powder prepared on the basis of a protein liquid feed subjected to conventional pasteurization and subsequent detached spray drying.

Moreover, the viscosities of the dispersions of the protein powders according to the invention were generally stable. If the viscosities decreased at all in response to the heating and cooling treatment described in the above, they did so by no more than 10% (cP at 50° C.)

This is in contrast to the dispersion of the protein powder prepared with the conventional heating process, which showed a drop in viscosity of more than 60% to a final level of less than 700 cP.

From the tests it appears that the viscosity is more stable than with conventional heat treatment before spray drying. The protein is highly activated and does not to any substantial extent loose viscosity when heat treated as described above.

### Example 2—Test with Soy Protein—Variation in Infusion Chamber Dimensions

The protein solid concentration was 8% (w/w) and the dispersion had a pH of 7. The dispersion was fed to the steam infusion nozzle at a rate of 120 kg/h and at a temperature of 50° C. Infusion chambers of varying length were tested; the distance between the end 104 of the first channel 101 of the infusion nozzle and the orifice 41 of the infusion chamber 103 was either 30 mm or 40 mm. A longer infusion chamber will, ceteris paribus, result in a longer residence time of the atomized protein in the infusion chamber. The nozzle pressure was 240 barg, and testing was performed at the following conditions:

TABLE 3
test parameters

| Test no. | 4 | 5 |
|---|---|---|
| Steam pressure (barg) | 8 | 8 |
| Steam flow (kg/h) (steam to | 105 | 105 |
| feed ratio 1:1.14) | | |
| Temperature (° C.) | 177 | 177 |
| Distance from end of first | 30 mm | 40 mm |
| channel of infusion nozzle to orifice | | |
| of infusion chamber | | |

The orifice of the infusion chamber had an opening diameter of 5 mm.

The resulting size of the final powders, measured as D₅₀ with a Malvern particle sizer, was 40 to 50 micron.

For evaluating the viscosity of the now functionalized protein, spray dried powders were redispersed in water at 12.5% w/w. The dispersions were high shear mixed for 1 minute, left to set for 6 h, whereafter viscosity was measured in a RVA apparatus (Rapid Visco Analyzer) at 50 rpm. For comparison, a 12.5% w/w dispersion was also prepared from a powder based on the same protein liquid feed but having been processed in a conventional manner, i.e. by conventional steam injection at 135° C. followed by a later, detached step of drying in a spray dryer as commonly known in the production process of soy protein isolates and/or concentrates.

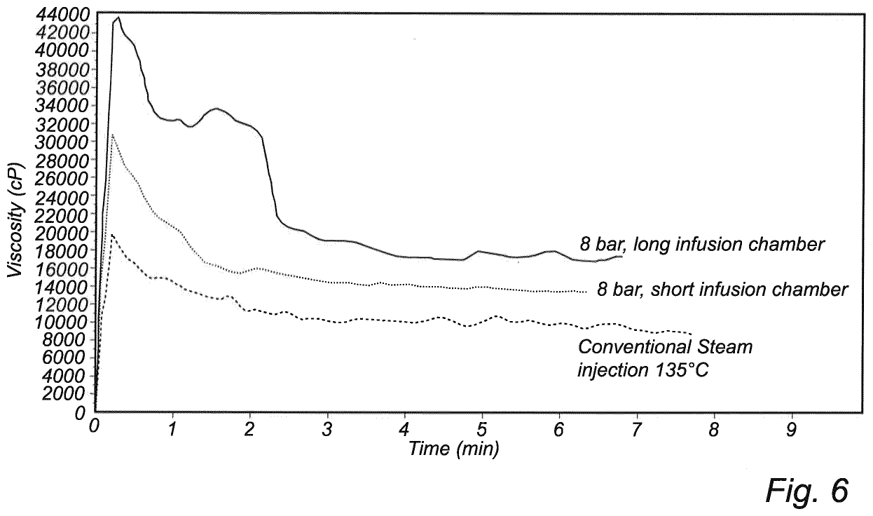

The viscosity of the respective dispersions was measured for at least 6 minutes using the standard procedure of the Rapid Visco Analyzer (PerkinElmer). The results appear from FIG. 6. Viscosities at 25° C. after 5 minutes were as follows:

TABLE 4
viscosity results

| | Viscosity at 25° C. |
|---|---|
| | (cP at 5 min) |
| Steam infusion nozzle | 13982 |
| 8 barg, short infusion chamber | |
| (Test 4) | |
| Steam infusion nozzle | 17781 |
| 8 barg, long infusion chamber | |
| (Test 5) | |
| Conventional steam injection | 9974 |

As appears, viscosities at 25° C., after 5 minutes, of almost 14000 cP or more were found for dispersions of protein powder prepared according to the invention. This is at least 40% higher than the viscosity found for the dispersion of protein powder prepared on the basis of a protein liquid feed subjected to conventional steam injection and subsequent detached spray drying.

### Example 3—Test with Pea Protein Isolate

The dispersion was fed to the steam infusion nozzle at a rate of 100-125 kg/h at 40° C. The cooking cap orifice was 6 mm (both a 4 and 5 mm cap resulted in less optimal atomization in this example).

The feed rate, steam flow and steam pressure were varied as well as the pH of the feed. Very fine powders were produced under the conditions shown below in table.

TABLE 5
test paramaters

| | Test 6 | Test 7 | Test 8 | Test 9 |
|---|---|---|---|---|
| Feed | 22 | 22 | 19 | 19 |
| concentration | | | | |
| (% DS) | | | | |
| pH of feed | 7 | 7 | 7 | 8.5 |
| Feed rate | 90 | 100 | 125 | 125 |
| kg/h | | | | |
| Nozzle | 195 | 240 | 230 | 230 |
| pressure (barg) | | | | |
| Steam flow | 90 | 70 | 60 | 62 |
| kg/h | | | | |
| Steam to | 1:1 | 1:1.42 | 1:2.08 | 1:2.02 |
| liquid feed | | | | |
| ratio | | | | |
| Cooking cap | 6 mm | 6 mm | 6 mm | 6 mm |
| orifice | | | | |
| Steam | 8 | 5.5 | 4.5 | 4.5 |
| pressure (barg) | | | | |
| Temperature | 164 | 152 | 144 | 144 |
| in nozzle (° C.) | | | | |
| Particle size | 45 | 50 | 50 | 51 |
| D₅₀ (micron) | | | | |

For evaluating the viscosity of the now functionalized protein, spray dried powders were redispersed in water at 20% w/w. The dispersions were mixed by hand and then heated from 50 to 100° C. in a RVA apparatus (Rapid Visco Analyzer) in the course of 3 minutes, kept at 100° C. for 2 minutes and then cooled to 50° C.

The viscosity at 50 rpm was measured before, during (not shown) and after heating and cooling using the standard procedure of the Rapid Visco Analyzer (PerkinElmer).

TABLE 6
viscosity results

| | Viscosity at 50° C. (cP) | Viscosity at 50° C. (cP) |
|---|---|---|
| | before heating | after heating |
| Test 6 | 578 | 1095 |
| Test 7 | 1400 | 610 |
| Test 10 | 432 | 564 |
| Test 11 | 1130 | 542 |

### DISCUSSION

While the results are not predictable the tests varying the different parameters indicate that increasing the solid concentration in the feed may affect the final viscosity.

In test 6, it is shown that a high feed concentration of 22% DS (dry substance) works in the method of the invention. In addition, it can be seen with comparative test 7 that the final viscosity profile is depending on the process conditions used (nozzle pressure, steam flow and steam pressure), such that the viscosity decreases after heating.

In tests 8 and 9 a variation with a lower steam to feed ratio and lower steam pressure than the other examples are shown, hence the results show the effect of varying these parameters on the final product. Hence, as can be seen from test 8, lowering the steam ratio also lowers the viscosity.

However, the viscosity is relatively stable such that heating does not further affect the initial viscosity.

In addition, tests 8 and 9 show the effect of varying pH of the feed hence by increasing pH higher than 8 the initial viscosity before heating is high while decreasing after heating, in this way it is possible to use pH to provide a different texture profile. For tests 6, 7, 8 and 9 cap orifices of 4 and 5 mm were also tested (not shown) but 6 mm gave the best results and is presently a preferred diameter for this application.

What is claimed is:

1. A method for drying non-mammalian protein, the method comprising:
providing a material comprising a non-mammalian protein;
extracting and separating protein from the material to obtain a protein enriched fraction of extracted protein, or otherwise obtaining a liquid, colloidal or semi-solid protein enriched fraction from the material;
providing a steam flow to a steam infusion nozzle mounted in a spray dryer, thereby generating a steam atmosphere in an infusion chamber of the steam infusion nozzle,
providing a protein liquid feed to the steam infusion nozzle;
introducing the protein liquid feed into the steam atmosphere of the infusion chamber of the steam infusion nozzle, at a pressure of at least 5 barg, thereby primary atomizing and heating the protein liquid feed in a process of primary atomization;
allowing the atomized, heated protein to directly enter a drying chamber of the spray dryer through an opening in the infusion chamber in a process of secondary atomization, the drying chamber being provided with a drying gas; and
drying the atomized, heated protein in the spray dryer to obtain a dried protein.

2. The method according to claim 1, wherein the protein liquid feed obtained is preheated to a temperature of 30 to 140° C. prior to being provided to the steam infusion nozzle.

3. The method according to claim 1, wherein the protein liquid feed is provided to the steam infusion nozzle at a protein solid concentration of 8 to 30% (w/w).

4. The method according to claim 1, wherein the pH of the protein liquid feed is in the range of 5 to 8.

5. The method according to any one of claims 1 to 3, wherein the pH of the protein liquid feed is in the range of 7 to 9.

6. The method according to claim 1, wherein the steam flow is provided to the steam infusion nozzle at a pressure of 1 to 16 barg.

7. The method according to claim 1, wherein the steam flow and the protein liquid feed are provided to the steam infusion nozzle in a weight proportion of steam to feed of 1:10 to 10:1.

8. The method according to claim 1, wherein the temperature in the infusion chamber during the process of primary atomization is in the range of 120 to 200° C.

9. The method according to claim 1, wherein the spray dryer is of a type, wherein the protein is further dried in an integrated or external fluid bed or in both an integrated and an external fluid bed.

10. The method according to claim 1, wherein a further processing of the dried protein is selected from agglomeration of the protein, lecithination of the protein, or a combination.

11. The method according to claim 1, wherein the means of extracting or separating protein from the material comprises one or more of alkaline extraction, membrane separation, ultrafiltration or a combination of micro- and ultrafiltration, or acid precipitation, whereby the protein enriched fraction is obtained by washing out undesirable small molecules from the material at a pH near the isoelectric point and keeping the precipitate or remaining protein enriched fraction.

12. The method according to claim 1, wherein the extracted protein is a pea protein; the protein liquid feed is adjusted to pH 6.5-8; the protein liquid feed is pre-heated to 40 to 80° C.; the protein liquid feed is provided to the steam infusion nozzle at a protein solid concentration of 18 to 22% (w/w); the steam flow and the protein liquid feed are provided to the steam infusion nozzle in a weight proportion of steam to liquid feed of 1:6 to 1:1; the protein liquid feed is introduced into the steam atmosphere of the infusion chamber of the steam infusion nozzle at a pressure of 150 to 270 barg; and the temperature in the infusion chamber during the process of primary atomization is in the range of 160 to 185° C.

13. Method according to claim 1, wherein the extracted protein is a soy protein; the protein liquid feed is adjusted to pH 6.5 to 8; the protein liquid feed is pre-heated to 45 to 65° C.; the protein liquid feed is provided to the steam infusion nozzle at a protein solid concentration of 8 to 14% (w/w); the steam flow and the protein liquid feed are provided to the steam infusion nozzle in a weight proportion of steam to liquid feed of 1:1.0 to 1:2; the protein liquid feed is introduced into the steam atmosphere of the infusion chamber of the steam infusion nozzle at a pressure of 180 to 300 barg; and the temperature in the infusion chamber during the process of primary atomization is in the range of 155 to 185° C.

14. System for drying a non-mammalian protein, the system comprising:
a spray dryer having a drying chamber, at least one drying gas inlet, and at least one product outlet;
at least one steam infusion nozzle configured for generating a protein liquid feed spray into the drying chamber of the spray dryer;
wherein the steam infusion nozzle comprises a first channel and a second channel, the first and second channels being in fluid communication with a steam infusion chamber included in the steam infusion nozzle, the first channel being connected to a supply of a liquid feed of the non-mammalian protein, the second channel being connected to a supply of steam, and the infusion chamber being provided with an opening facing the drying chamber of the spray dryer.

15. A system according to claim 14, wherein the opening of the infusion chamber has a diameter of 2 to 8 mm.

16. A dried, non-mammalian protein powder being functionalized or showing bacteria or spore counts reduced to a stipulated level, obtainable by the method of claim 1.

17. A functionalized, dried, non-mammalian protein powder according to claim 16, wherein the protein is pea protein, and wherein a dispersion of 14% (w/w) of the powder in water shows, after being heated in the course of 3 minutes from 50 to 100° C., kept at 100° C. for 2 minutes, and then cooled to 50° C., a level of viscosity at 50° C. of more than 2500 cP (mPa*s).

18. A functionalized, dried, non-mammalian protein powder according to claim 16, wherein the protein is soy protein, and wherein a dispersion of 12.5% (w/w) of the powder in water, 5 minutes after preparation of the dispersion, shows a level of viscosity at 25° C. of more than 13000 cP (mPa*s).

19. The method according to claim 1, wherein the non-mammalian protein is a native non-mammalian protein.

20. The method according to claim 1, further comprising dispersing, and pH adjusting the protein enriched fraction of extracted protein or the otherwise obtained protein enriched fraction to obtain the protein liquid feed, wherein the pH adjustment is above an isoelectric point of the protein.

21. The method according to claim 3, wherein the protein liquid feed is provided to the steam infusion nozzle at a protein solid concentration of 15 to 25% (w/w).

22. The method according to claim 3, wherein the protein liquid feed is provided to the steam infusion nozzle at a protein solid concentration of 17 to 22% (w/w).

23. The method according to claim 7, wherein the steam flow and the protein liquid feed are provided to the steam infusion nozzle in a weight proportion of steam to feed of 1:7 to 3:1.

24. The method according to claim 7, wherein the steam flow and the protein liquid feed are provided to the steam infusion nozzle in a weight proportion of steam to feed of 1:6 to 2:1.

25. The method according to claim 7, wherein the steam flow and the protein liquid feed are provided to the steam infusion nozzle in a weight proportion of steam to feed of 1:3 to 1:2.

26. The method according to claim 12, wherein the steam flow and the protein liquid feed are provided to the steam infusion nozzle in a weight proportion of steam to liquid feed of 1:3 to 1:1.5.

27. The method according to claim 12, wherein the steam flow and the protein liquid feed are provided to the steam infusion nozzle in a weight proportion of steam to liquid feed of 1:2 to 1:1.

28. A system according to claim 15, wherein the opening of the infusion chamber has a diameter of 3 to 7 mm.

29. A system according to claim 15, wherein the opening of the infusion chamber has a diameter of 6 mm.
